(19) United States
(12) Patent Application Publication
(10) Pub. No.: US 2005/0159788 A1
(43) Pub. Date: Jul. 21, 2005

(54) Transmurality assessment apparatus and methods

(76) Inventors: David C Kress, Franklin, WI (US); David P. Krum, Muskego, WI (US); John W. Hare, Mequon, WI (US); Jashir Sra, Pewaukee, WI (US)

Correspondence Address: MICHAEL BEST & FRIEDRICH, LLP, ONE SOUTH PINCKNEY STREET, P O BOX 1806, MADISON, WI, 53701, US

(21) Appl. No.: 10/517,469

(22) PCT Filed: Jun. 16, 2003
(86) PCT No.: PCT/US03/18464

Related U.S. Application Data
(60) Provisional application No. 60/389,016, filed on Jun. 14, 2002.

(51) Int. Cl. A61N 1/362

(52) U.S. Cl. 607/32; 607/122

(57) ABSTRACT

A transmurality evaluation apparatus or probe, method for manufacturing a transmurality evaluation apparatus of probe, and method for assessing the transmurality of a lesion. In one aspect, the transmurality evaluation apparatus or probe generally includes a handheld elongate housing (30), wiring (20) having a first end and a second end, at least one tissue contact (26) electrically coupled to the wiring first end, and a terminal connector (28) electrically coupled to the wiring second end. In another aspect, the method for assessing the transmurality of a lesion generally includes establishing a first electrogram of the first region of tissue, ablating a second region of tissue to form an ablated lesion, establishing a second electrogram of the first region of tissue, and comparing the first and second electrograms to assess transmurality of the ablated lesion.

### RELATED APPLICATION

This is a continuation of Provisional Application Ser. No. 60/389,016, filed on Jun. 14, 2002.

### BACKGROUND OF THE INVENTION

This invention relates to assessing transmurality of a lesion. More specifically, this invention relates to assessing transmurality of an ablated lesion.

The heart includes a number of pathways that are responsible for the propagation of signals necessary to produce continuous, synchronized contractions (i.e., sinus rhythm). Each contraction cycle in sinus rhythm begins in the right atrium where a sinoatrial node initiates an electrical impulse. This impulse then spreads across the right atrium to the left atrium, stimulating the atria to contract. The chain reaction continues from the atria to the ventricles by passing through a pathway known as the atrioventricular (AV) node or junction, which acts as an electrical gateway to the ventricles. The AV junction delivers the signal to the ventricles while also slowing it, so the atria can relax before the ventricles contract.

Disturbances in the heart's electrical system may lead to various problems that can cause the heart to beat irregularly, too fast or too slow. Irregular heart beats, or arrhythmia, are caused by physiological or pathological disturbances in the discharge of electrical impulses from the sinoatrial node, in the transmission of the signal through the heart tissue, or spontaneous, unexpected electrical signals generated within the heart from independent foci. One type of arrhythmia is tachycardia, which is an abnormal rapid beating of the heart. There are several different forms of atrial tachycardia, including atrial fibrillation and atrial flutter. With atrial fibrillation, instead of a single beat, numerous electrical impulses are generated by depolarizing tissue at one or more locations in the atria (or possibly other locations). Atrial fibrillation may be focal in nature, caused by the rapid and repetitive firing of an isolated center within the atrial cardiac muscle tissue. These isolated centers or independent foci, defined by regions exhibiting a consistent and centrifugal pattern of electrical activation, may act as either a trigger of atrial fibrillatory paroxysmal or may even sustain the fibrillation.

The precise cause of atrial fibrillation, and in particular the depolarizing tissue causing “extra” electrical signals, is currently unknown. As to the location of the depolarizing tissue, it is generally agreed that the undesired electrical impulses often originate in the left atrial region of the heart. Recent studies have expanded upon this general understanding, suggesting that nearly 90% of these “focal triggers” or electrical impulses are generated in one (or more) of the four pulmonary veins (PV) extending from the left atrium. In this regard, as the heart develops from an embryotic stage, left atrium tissue may grow or extend a short distance into one or more of the PVs. It has been postulated that this tissue may spontaneously depolarize, resulting in an unexpected electrical impulse(s) propagating into the left atrium and along the various electrical pathways of the heart.

A variety of different atrial fibrillation treatment techniques are available, including drugs, surgery, implants, and surgical and catheter-based ablation. Surgical and catheter-based treatments can cure the problem by ablating the abnormal tissue or accessory pathway responsible for the atrial fibrillation. Ablation is the process of removing, especially by cutting, abrading, or evaporating. Ablation treatments may be performed either from within the chambers of the heart (i.e., endocardial ablation) using endovascular devices (e.g. catheters) introduced through arteries or veins, or from outside the heart (i.e., epicardial ablation) using devices introduced into the chest through surgical incisions or via a sternotomy. Ablation treatments rely on the application of various destructive energy sources to the target tissue, including direct current electrical energy, unipolar radiofrequency electrical energy, bipolar radiofrequency electrical energy, diode laser energy, microwave energy, high-intensity focused ultrasound, argon cryothermia, and the like. Ablation devices are used to create elongated transmural lesions (i.e., lesions extending through a sufficient thickness of the myocardium to block electrical conduction) which form the boundaries of the conductive corridors in the atrial myocardium. Transmural ablation can be performed on the beating heart without the use of cardiopulmonary bypass.

Due to the relatively small thickness of atrial tissue formed within a PV, it is possible that ablation of this tissue may in fact cause stenosis in the PVs. Even further, other vital bodily structures are directly adjacent each PV. These structures may be undesirably damaged when ablating within a PV.

In light of the above, an alternative technique has been suggested whereby a continuous ablation lesion pattern is formed in the left atrium wall about the ostium associated with the PV in question. In other words, the PV is electrically isolated from the left atrium by forming an ablation lesion pattern that surrounds the PV ostium. As a result, any undesired electrical impulse generated within the PV could not propagate into the left atrium, thereby eliminating unexpected atrial contraction.

### SUMMARY OF THE INVENTION

Briefly, in one aspect, the present invention is generally directed to a transmurality evaluation apparatus or probe generally comprising a handheld elongate housing having a first end and a second end and defining an interior passage therebetween, wiring having electrically coupled first and second ends and positioned within the housing passage and passing therethrough, at least one tissue contact electrically coupled to the first end of the wiring adjacent the first end of the housing, and a terminal connector electrically coupled to the second end of the wiring.

In another aspect, the present invention is generally directed to a method for assessing the transmurality of a lesion generally comprising providing a probe adapted to sense an electrical signal from tissue, contacting a first region of tissue with the probe, establishing a first electrogram of the first region of tissue, ablating a second region of tissue to form an ablated lesion, contacting the first region of tissue with the probe, establishing a second electrogram of the first region of tissue, and comparing the first and second electrograms to assess transmurality of the ablated lesion.

In another aspect, the present invention is generally directed to a method for manufacturing a transmurality evaluation apparatus generally comprising providing a handheld elongate housing having a first end and a second end and defuiing an interior passage therebetween, positioning wiring having a first end electrically coupled to a second end in the interior passage to electrically couple the housing first end to the housing second end, electrically coupling a tissue contact to the wiring first end adjacent the housing first end, and electrically coupling a terminal connector to the wiring second end.

Other features and aspects of the invention will become apparent to those skilled in the art upon review of the following detailed description, claims, and drawings, wherein like elements have like numerals throughout the drawings.

### BRIEF DESCRIPTION OF THE DRAWINGS

The present invention is further described with reference to the accompanying drawings, which show an exemplary embodiment of the present invention, and wherein like numbers represent like elements throughout. However, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention. Also, it is understood that the phraseology and terminology used herein is for the purpose of description and should not be regarded as limiting. Certain terminology, for example, “distal,” “proximal,” “top,” “bottom,” “right,” “left,” “front,” “frontward,” “forward,” “back,” “rear,” and “rearward,” may be used in the following description for relative descriptive clarity only and is not intended to be limiting.

FIG. 1A is a perspective view of a transmurality evaluation probe embodying the present invention having a housing, wiring, tissue contact and terminal connector.

FIG. 1B is a close-up partial section view of the probe of FIG. 1.

FIG. 1C is a close-up perspective view of a probe according to a second embodiment of the present invention.

FIG. 1D is a close-up perspective view of a probe according to a third embodiment of the present invention.

FIG. 1E is a close-up perspective view of an alternative embodiment of the terminal connector of FIG. 1A.

FIG. 2A is a preablation electrogram and a postablation electrogram of the right superior pulmonary vein.

FIG. 2B is a preablation electrogram and a postablation electrogram of the left superior pulmonary vein.

FIG. 2C is a preablation electrogram and a postablation electrogram of the right inferior pulmonary vein.

FIG. 2D is a preablation electrogram and a postablation electrogram of the left inferior pulmonary vein.

FIG. 3A is an electrocardiogram and an electrogram of the right superior pulmonary vein recorded simultaneously prior to ablation.

FIG. 3B is an electrocardiogram and an electrogram of the right superior pulmonary vein recorded simultaneously after ablation.

FIG. 4A is an electrocardiogram and an electrogram of the right inferior pulmonary vein recorded simultaneously prior to ablation.

FIG. 4B is an electrocardiogram and an electrogram of the right inferior pulmonary vein recorded simultaneously after ablation.

FIG. 5A is an electrogram of the right superior pulmonary vein and tracings of limb leads I and II recorded simultaneously prior to ablation.

FIG. 5B is a chart of the tracings of FIG. 5A after a first ablation.

FIG. 5C is a chart of the tracings of FIG. 5A after a second ablation.

FIG. 5D is a chart of the tracings of limb leads I and II of FIG. 5A recorded simultaneously with electrograms 1 and 2 of the right atrial appendage, illustrating sinus rhythm.

### DETAILED DESCRIPTION OF THE PREFERRED EMBODIMENT

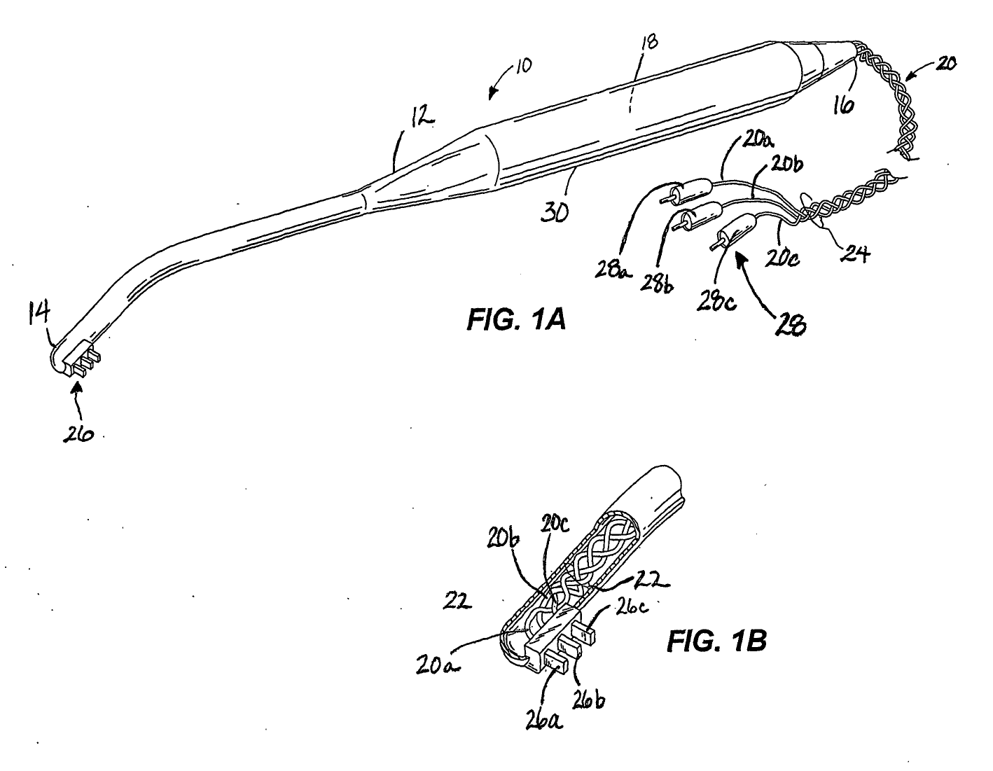

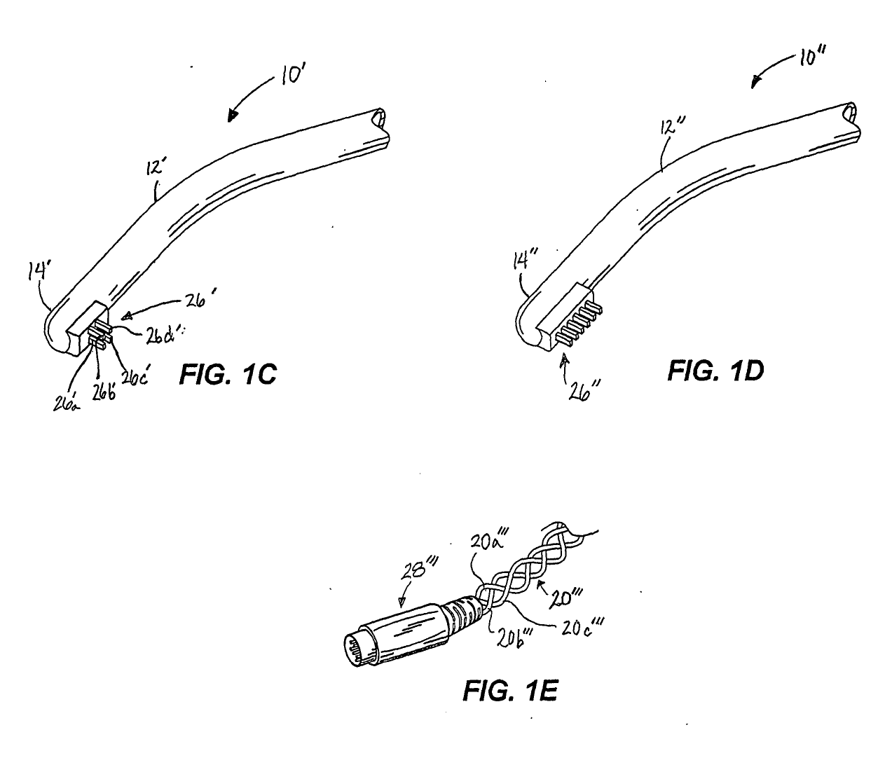

FIGS. 1A and 1B illustrate one embodiment of a transmurality evaluation apparatus or probe 10 of the present invention. The apparatus or probe 10 includes a handheld elongate housing 12 having a distal end 14 and a proximal end 16 and further defines an interior passage 18. Probe 10 further includes insulated wiring 20 having a first end 22 and a second end 24 and passing through the interior passage 18. Probe 10 further includes at least one tissue contact, collectively referred to as 26, electrically coupled to the wiring first end 22 and adjacent the distal end 14. Probe 10 further includes at least one terminal connector 28 electrically coupled to the wiring second end 24.

The handheld elongate housing 12 of the embodiment illustrated in FIGS. 1A and 1B is preferably formed of a molded polymer. Housing 12 can be formed of a variety of opaque, translucent or transparent materials including, but not limited to, polystyrene, polyethylene, polypropylene, polyethylene terephthalate, acrylonitrile butadiene styrene, ceramic, etc., and combinations thereof. Interior passage 18 of the embodiment illustrated in FIGS. 1A and 1B has a generally narrow circular cross-section adjacent distal end 14 that gradually increases in diameter to a thicker, generally more square cross-section adjacent a hand-piece 30, and finally tapers to a generally narrow circular portion adjacent proximal end 16. Hand-piece 30 can have an exterior texture or be coated with any of a variety of gripping materials to aid in manipulation of probe 10. Furthermore, probe 10 can have a variety of cross-sectional shapes without departing from the scope of the present invention.

Probe 10 can be of any size, as determined by the specific application of probe 10. For example, at least a portion of probe 10 (e.g., a portion adjacent distal end 14) can be relatively long and narrow so as to allow its placement through a small thoracic incision or a thoracic surgical port as would be prepared during minimally invasive thoracic surgery. Furthermore, probe 10 can be flexible to a varying degree and therefore formed of a variety of materials, the flexibility and materials chosen based on the desired application.

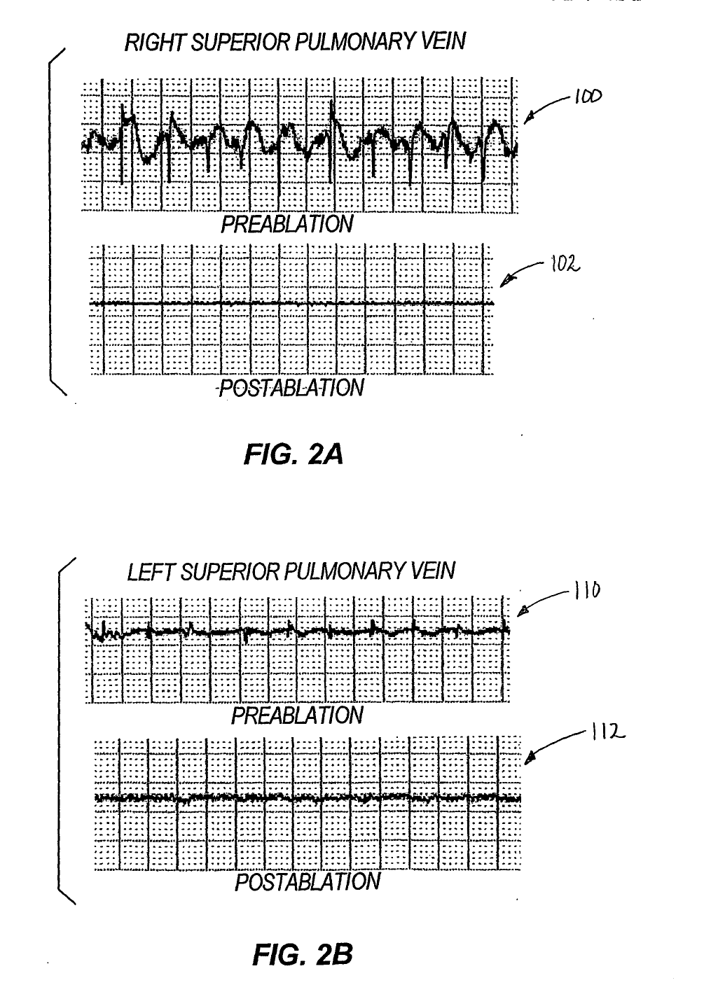

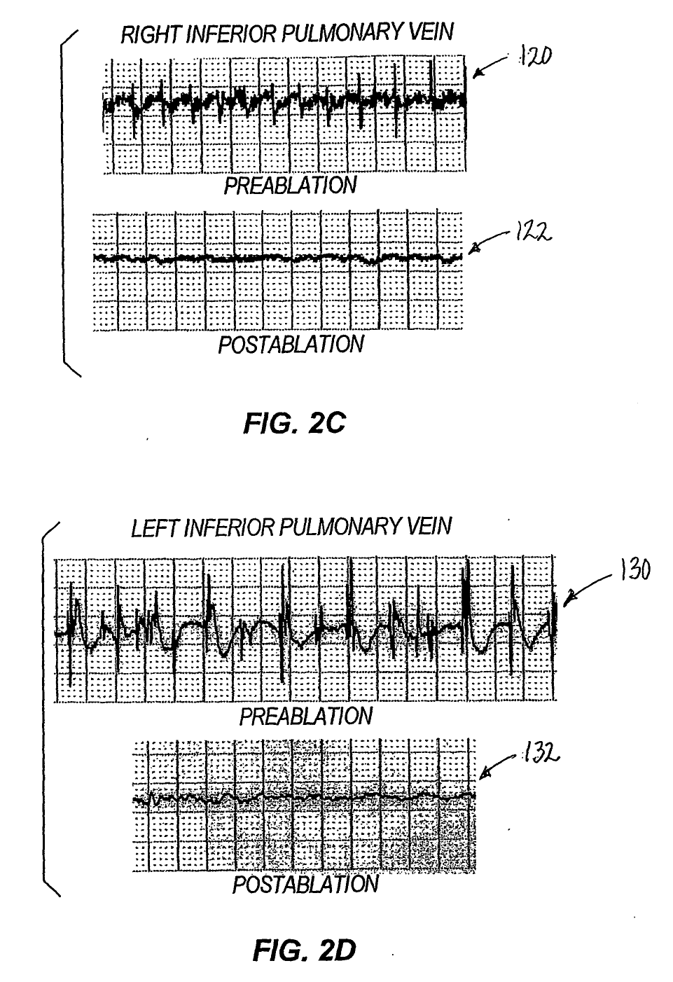

Wiring 20 of the embodiment illustrated in FIGS. 1A and 1B includes at least one insulated wire and as many as appropriate. In addition, wiring 20 is shown broken in FIG. 1A to signify that wiring 20 can be of any desired length, as determined by the specific application of probe 10. As illustrated in FIGS. 1A and 1B, probe 10 preferably includes a first wire 20a, a second wire 20b and a third wire 20c wound around each other to minimize electrical interference. As illustrated in FIG. 2, probe 10 further includes a first tissue contact 26a electrically coupled to first wire 20a, a second tissue contact 26b electrically coupled to second wire 20b, and a third tissue contact 26c electrically coupled to third wire 20c. Probe 10 includes tissue contacts 26 to measure the difference in potential between any two points that represents the work involved or the energy released in the transfer of a unit quantity of electricity from one point to the other. Particularly, this potential difference can be measured in volts and displayed accordingly. Therefore, probe 10 can have at least two tissue contacts 26 such that the potential difference between any two tissue contacts 26 can be measured. For example, probe 10 is shown as having three tissue contacts 26a, 26b, 26c. Accordingly, probe 10 can measure three potential differences, because probe 10 can measure the potential difference between any two of tissue contacts 26a, 26b, 26c (i.e., between 26a and 26b, 26a and 26c, and/or 26b and 26c). Since wires 20a, 20b, 20c are electrically coupled to tissue contacts 26a, 26b, 26c, respectively, each of the wires 20a, 20b, 20c can be electrically coupled directly or via a variety of adapters or connectors to any type of chart recorder that can display potential difference. That is, any two of wires 20a, 20b, 20c can be electrically coupled to a device that displays potential difference in real time, and the potential difference between all pairs can then be examined. Alternatively, all three wires 20a, 20b, 20c can be electrically coupled to a device capable of displaying multiple potential differences at once, such that the potential difference between all pairs of tissue contacts 26 can be visualized simultaneously in real time.

FIGS. 1C-1E illustrate alternative embodiments for tissue contacts and terminal connectors for use with a probe according to the present invention. FIG. 1C illustrates a probe 10′ according to a second embodiment of the present invention. Probe 10′ includes a handheld elongate housing 12′ having a distal end 14′. Probe 10′ further includes four tissue contacts 26a′, 26b′, 26c′ and 26d′ (collectively referred to as 26′) adjacent distal end 14′, arranged in two lateral rows and two longitudinal columns. Each tissue contact 26a′, 26b′, 26c′, 26d′ is electrically coupled to a wire as described above with respect to FIGS. 1A and 1B (not shown in FIG. 1C for clarity), and the potential difference between any two of the tissue contacts 26′ can be measured as described above. There are six potential differences (i.e., between 26a′ and 26b′, 26a′ and 26c′, 26a′ and 26d′, 26b′ and 26c′, 26b′ and 26d′, and/or 26c′ and 26d′) that can be measured individually or simultaneously, as described above. Tissue contacts 26′ can be arranged or configured in any number of rows and columns desired. Providing different tissue contact configurations allows measurement of the potential difference between points on a wide variety of tissues, such as short, thin and/or incomplete muscles. Furthermore, increasing the total number of tissue contacts 26′ allows the generation of more signals and a more accurate reading of potential difference in a particular tissue at a given time.

FIG. 1D illustrates a probe 10″ according to a third embodiment of the present invention. Probe 10″ includes a handheld elongate housing 12″ having a distal end 14″. Probe 10″ further includes six tissue contacts, collectively referred to as 26″. Similar to the first and second embodiments, tissue contacts 26″ can each be electrically coupled to a wire (not shown in FIG. 1D for clarity) such that the potential difference between any two tissue contacts 26″ can be measured individually or simultaneously. By providing six tissue contacts arranged generally linearly, the tissue contacts can make up for any gaps or breaks in a particular tissue and can increase the accuracy of the potential difference read in a particular tissue, as explained above. Any number of tissue contacts arranged in a variety of configurations can be used without departing from the spirit and scope of the present invention. Furthermore, tissue contacts 26, 26′, 26″ are illustrated as being generally parallelepiped-shaped. However, tissue contacts 26, 26′, 26″ can instead be cylindrical, conical, pyramidal, or have any other shape necessary to measure the potential difference between two contact points.

Referring again to probe 10 of FIG. 1A, first wire 20a is electrically coupled to a first terminal connector 28a, second wire 20b is electrically coupled to a second terminal connector 28b, and third wire 20c is electrically coupled to a third terminal connector 28c. Terminal connectors 28 of FIG. 1A are adapted to be plugged into at least one of an electrocardiogram strip recorder, an implantable cardiac defibrillator (ICD)/pacemaker programmer, telemetry recording device, an oscilloscope, or any other chart recorder capable of recording potential difference, and specifically, voltage (i.e., potential difference measured in volts).

FIG. 1D illustrates a terminal connector 28′″ according to an alternative embodiment of the present invention. Terminal connector 28′″ is illustrated as being electrically coupled to wiring 20′″, and specifically to three wires 20a′″, 20b′″ and 20c′″. Terminal connector 28′″ can instead be electrically coupled to two wires, four wires, six wires, and so on, depending on which type of probe and tissue contacts of the present invention are used. Terminal connector 28′″ is adapted to be electrically coupled to any of the potential difference recording equipment mentioned above. Other types of male or female terminal connectors or combinations of terminal connectors are possible and are within the scope of the present invention.

Probes 10 can be manufactured generally by creating or providing handheld elongate housing, positioning wiring 20 in interior passage 18 of housing 12 to electrically couple distal end 14 to proximal end 16, electrically coupling a tissue contact 26 to wiring first end 22 adjacent distal end 14, and electrically coupling a terminal connector 28 to wiring second end 24. It should be readily apparent to those of ordinary skill in the art that probe 10 of the present invention can be manufactured using a variety of materials and techniques but generally follows the process laid out above. An exemplary embodiment of the present invention includes a housing 12 formed by the body of a commercially available YANKAUER® brand sucker (“Medivac Yankauer Sucker with Tapered Bulbous Tip,” Allegiance Corporation, McGaw Park, Ill. 60085) having a distal end 14, a proximal end 16 and further defining an interior passage 18. To manufacture the exemplary embodiment of probe 10, a distal tip adjacent distal end 14 of the YANKAUER® brand sucker is removed to provide a distal opening to which at least one tissue contact 26 is attached. Two to six or more insulated wires 20, each having a first end 24 and a second end 26 are passed through the YANKAUER® brand sucker to provide electrical continuity therethrough. The wires 20 are wound around each other to minimize electrical interference. Two to six metal tissue contacts 26 are then soldered to the first ends 22 of the two to six wires 20, respectively, adjacent the YANKAUER® brand sucker modified distal end 14. Adhesive is used to attach at least one tissue contact 26 in place at distal end 14 of the YANKAUER® brand sucker body. Second ends 24 of the two to six wires 20 are attached to two to six terminal connectors 28, respectively. Probes 10′ and 10″ can be manufactured using similar methods to those described above, and it will be readily apparent to one of ordinary skill in the art that other probes having different configurations, shapes and assemblies within the scope of the present invention can be manufactured or assembled using these methods.

Probes 10, 10′, 10″ can be used to detect electrical activity in tissues, and therefore can assess the transmurality of a lesion. Said another way, probes 10, 10′, 10″ can be used to determine whether regions of tissue are electrically connected or isolated from one another. For example, if is desired to electrically isolate a first region of tissue from a second region of tissue, probes 10, 10′, 10″ can be used to establish a baseline electrogram of the first region of tissue. Various surgical or ablative treatments can be used to create a lesion between the first and second regions of tissue or on one of the first or second regions of tissue to electrically isolate the first and second regions of tissue. Once a lesion has been created to attempt to electrically isolate the first and second regions of tissue, probes 10, 10′, 10″ can be used to establish a second electrogram of the first region. The second electrogram can then be compared to the baseline electrogram to determine whether the first region of tissue has been electrically isolated from the second region of tissue, that is, whether the lesion is transmural. Touch-up surgical or ablative treatments can be performed until a transmural lesion is confirmed with a relatively silent electrogram of the first region of tissue. One exemplary application of probes 10, 10′, 10″ is the use of probes 10, 10′, 10″ to determine whether the pulmonary veins (PVs) have been electrically isolated from the left atrium following surgical ablation of atrial fibrillation. More specifically, probes 10, 10′, 10″ can be used to epicardially assess the transmurality of a lesion that has been created to electrically isolate the PVs from the left atrium. In humans, oxygenated blood is transported from the lungs to the left atrium of the heart via four PVs, a right superior pulmonary vein (RSPV), a right inferior pulmonary vein (RIPV), a left superior pulmonary (LSPV), and a left inferior pulmonary vein (LIPV). Each PV has a circumferential muscle sleeve adjacent the left atrium that may be responsible for producing aberrant electrical impulses that give rise to atrial fibrillation and atrial flutter, as discussed above. Various lesion patterns applied to the left atrium (or the PVs) have proven to be successful in electrically isolating the PV muscle sleeves from the left atrium. Examples of successful lesion patterns and the methodologies used are described in greater detail in “Validation of a left atrial lesion pattern for intraoperative ablation of atrial fibrillation,” by David C. Kress, et al., published in the Annals of Thoracic Surgery, 73: 4, 1160-1168 (April 2002); “Radiofrequency ablation of atrial fibrillation during mitral valve surgery,” by David C. Kress et al., published in Semin Thorac Cardiovasc Surg. 2002 July; 14(3): 210-8; and “Surgical Ablation of Atrial Fibrillation: Is there a “best” lesion pattern?,” by David C. Kress, MD, published in Heart Surgery Forum Reviews, 1: 1 (2002)), which are herein incorporated by reference. To assess the transmurality of an ablated lesion, and thus the effective electrical isolation of a PV from the left atrium, probe 10, 10′ or 10″ is used to establish a baseline or preablation electrogram for the PV. A lesion is then created endocardially or epicardially with a first ablation treatment using any of a variety of commercially-available cardiac ablation probes. After the first ablation treatment, probe 10, 10′ or 10″ is used to establish a postablation electrogram for the PV. The postablation electrogram for the PV is compared to the baseline electrogram. By comparing the postablation electrogram to the preablation electrogram, one of ordinary skill in the art can determine whether left atrial electrical activity is still being transmitted onto the PV muscle sleeve, and therefore, assess the transmurality of the lesion. If left atrial electrical activity is still sensed in the PV muscle sleeve, a second ablation treatment is applied. This process continues until a transmural ablated lesion has been created, signifying substantial electrical isolation of the PV from the left atrium.

Alternatively, depending on the lesion pattern created, probes 10, 10′, 10″ can be used to detect electrical isolation of the PVs from the left atrium by sensing electrical activity on at least a portion of the left atrium. For example, one lesion pattern that can be used to isolate the PVs from the left atrium forms a circle on the left atrium that encapsulates the ostia for the PVs. Therefore, probe 10, 10′ or 10″ can be used to contact a region on the left atrium within the ablated circle to test the isolation of all PVs at once. Of course, if electrical activity is sensed, other areas of the left atrium or PVs will need to be tested to locate the nontransmural portion of the lesion or the gaps.

Alternatively, probes 10, 10′, 10″ can be adapted to deliver a pacing signal to a first region of tissue and/or sense electrical activity of a second region of tissue opposite a lesion to detect whether any of the paced signal is transmitted across the lesion to assess the transmurality of the lesion. More specifically, probes 10, 10′, 10″ can be adapted to epicardially pace across the lesion to assess the transmurality of the lesion.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described herein are further intended to explain best modes known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

### EXAMPLE 1

PV electrograms were recorded preablation and postablation to assess circumferential transmurality and lack of gaps. A gap in a lesion refers to a complete lack of lesion in a particular location and can be created when there is not at least a slight overlap is successive lesions. Tracings were made using a probe of the present invention electrically coupled to a standard ICD/pacemaker programmer and chart recorder. FIGS. 2A-2D illustrate and compare the preablation and postablation PV electrograms. FIG. 2A illustrates a first, or baseline, electrogram 100 of the RSPV that was established by epicardially contacting the RSPV with the probe 10 prior to ablation. Accordingly, electrogram 100 is labeled “preablation.” Electrogram 100 illustrates rhythmic atrial depolarization that was being captured by the RSPV, and which signified that electrical activity from the left atrium was being transmitted onto the muscle sleeve of the RSPV. FIG. 2A further illustrates a second electrogram 102 of the RSPV that was established by epicardially contacting the RSPV with the probe after ablation (i.e., postablation). Compared to baseline electrogram 100, electrogram 102 signified relative electrical silence, thereby signifying that the RSPV had been effectively electrically isolated from the left atrium. Furthermore, electrogram 102 included no spontaneous depolarization voltage peaks, which implied that there were no independent, spontaneously depolarizing foci within the RSPV at that time. Similarly, FIG. 2B illustrates a preablation electrogram 110 of the LSPV that demonstrated the transmission of atrial electrical activity onto the muscle sleeve of the LSPV, and a postablation electrogram 112 of the LSPV. Postablation electrogram 112 demonstrated that a transmural and gapless lesion had been created, and that the LSPV had been effectively electrically isolated from the left atrium. The low amplitude of left atrial depolarization captured in preablation electrogram 110 of the LSPV implied that the LSPV had a relatively short and/or thin muscle sleeve. FIG. 2C illustrates a preablation electrogram of the RIPV and a postablation electrogram of the RIPV. Similar to the electrograms illustrated in FIGS. 2A and 2B, the comparison of preablation and postablation electrograms 120, 122 of the RIPV signified effective electrical isolation of the RIPV from the left atrium and a transmural and gapless lesion. FIG. 2D illustrates a preablation electrogram 130 of the LIPV and a postablation electrogram 132 of the LIPV, which similarly illustrated an effective electrical isolation of the RIPV from the left atrium and a transmural and gapless lesion. Ablation was performed by applying radiofrequency electrical energy epicardially to the left atrium in a lesion pattern described in greater detail in “Radiofrequency and Microwave Ablation for Atrial Fibrillation,” Chapter 30 written by David C. Kress, MD, of book entitled, “Advanced Therapy in Cardiac Surgery,” Ed. 2, to be published by B.C. Decker, Inc., Ontario in January 2003, which is incorporated herein by reference. Recording of second electrograms 102, 112, 122, 132 can be recorded during the ablation procedure as the left atrium is ablated adjacent particular PV ostia or after an entire first ablation treatment has been applied to create the desired lesion pattern.

### EXAMPLE 2

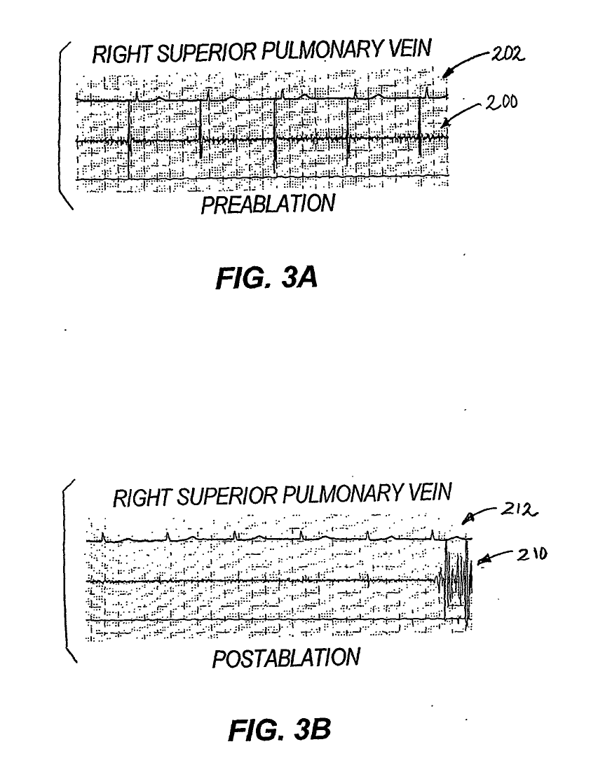

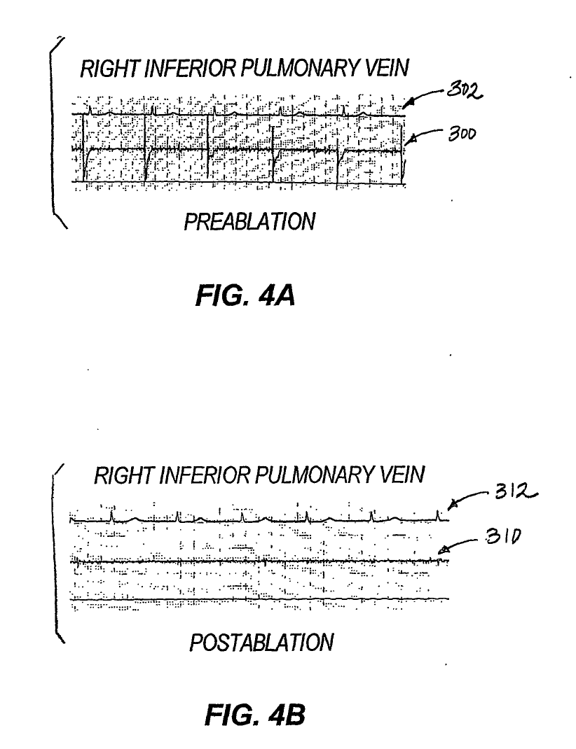

Following methods similar to the methods described above, an electrocardiogram (ECG) was recorded simultaneously with preablation and postablation electrograms for the RSPV and the RIPV using a probe of the present invention electrically coupled to a standard ICD programmer chart recorder. FIG. 3A illustrates a preablation electrogram 200 for the RSPV recorded simultaneously with an ECG 202. Pairing preablation electrogram 200 with ECG 202 confirmed that left atrial activity was being transmitted onto the muscle sleeve of the RSPV, because the peaks in the RSPV preablation electrogram 200 corresponded to ECG p-waves. As illustrated in FIG. 3B, a RSPV postablation electrogram 210 was recorded simultaneously with an ECG 212 after ablation was complete, which demonstrated that the p-wave-correlated peaks from the RSPV preablation electrogram 200 had disappeared. Furthermore, any small peaks that arose in the RSPV postablation electrogram 210 were identified as being farfied QRS signal by comparison with the ECG 212. Therefore, pairing the ECG signals 202, 212 with RSPV preablation and postablation electrograms 200, 210, respectively, effectively demonstrated that the RSPV had been electrically isolated from the left atrium and that a transmural and gapless lesion had been created circumferentially in the muscle sleeve of the RSPV. Following the same methods with the RIPV, a preablation electrogram 300 was simultaneously recorded with an ECG 302, as illustrated in FIG. 4A, and a postablation electrogram 310 was simultaneously recorded with an ECG 312, as illustrated in FIG. 4B. Pairing RIPV preablation electrogram 300 with ECG 302 illustrated that left atrial activity was being transmitted onto the muscle sleeve of the RIPV, because the peaks in RIPV preablation electrogram 300 correspond with the p-waves of ECG 302. As evident from FIG. 4B, the RIPV was electrically isolated from the left atrium, and a transmural lesion was confirmed.

### EXAMPLE 3

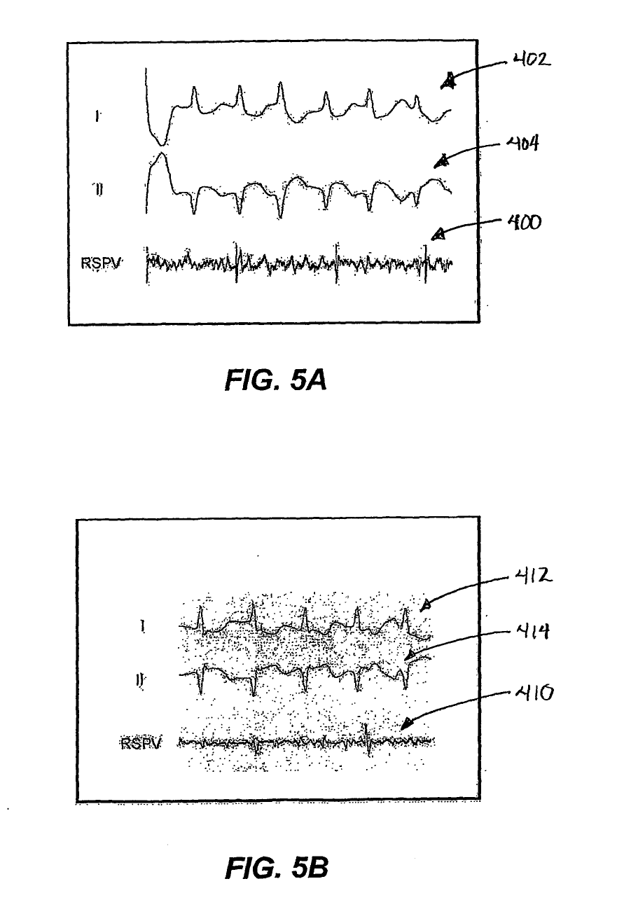

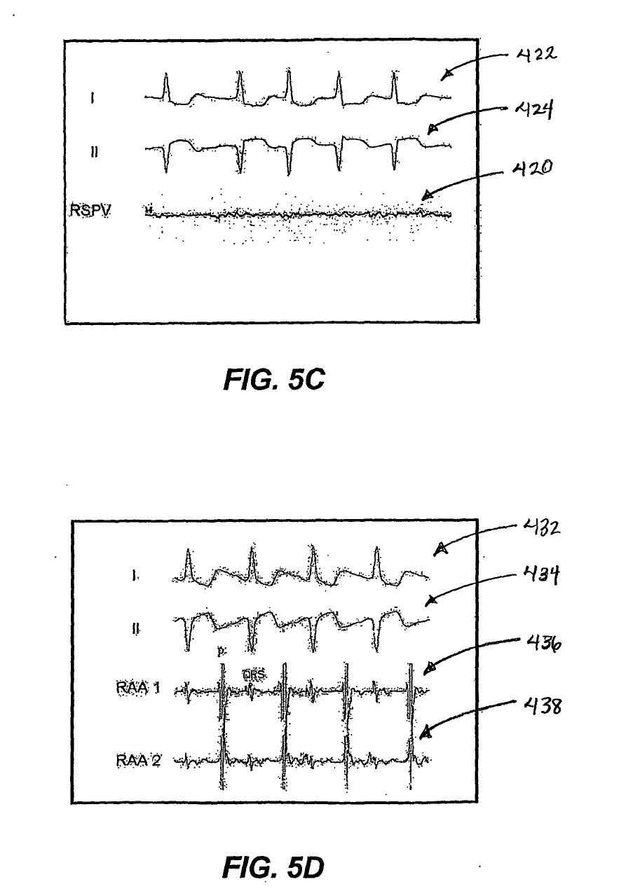

A baseline RSPV muscle sleeve electrogram 400 exhibiting transmitted left atrial activity was recorded by with a bipolar probe according to the present invention electrically coupled to a strip recorder with reference limb lead I tracing 402 and reference limb lead II tracing 404, as illustrated in FIG. 5A. An ablation treatment was then applied that created a lesion pattern for surgical ablation of atrial fibrillation. A postablation RSPV muscle sleeve electrogram 410 was recorded using the same bipolar probe with reference limb lead I tracing 412 and reference limb lead II tracing 414, as illustrated in FIG. 5B. Postablation RSPV muscle sleeve electrogram 410 included persistent low amplitude atrial signals (as determined by comparing postablation electrogram 410 with reference limb lead tracings 412, 414), which indicated non-transmural lesions or gaps. A touch-up ablation treatment was then applied. A second postablation RSPV muscle sleeve electrogram 420 was recorded with two reference limb lead tracings 422, 424, as illustrated in FIG. 5C. Postablation RSPV muscle sleeve electrogram 420 exhibited primarily far-field QRS activity, which signified transmural lesions between the left atrium and the RSPV. The transmurality of the lesions was confirmed by recording right atrial appendage electrograms 436, 438 with reference limb lead tracings 432, 434, as illustrated in FIG. 5D. Right atrial appendage electrograms 436, 438 show a patient in sinus rhythm following the ablation treatment, which confirmed the transmurality of the lesions. The ablation treatments included the application of microwave energy using an AFx Flex-4 brand microwave ablation probe and Series 1000 generator, as described in greater detail in a draft manuscript entitled “Off pump epicardial microwave ablation of fibrillation: Technique and early results,” authors David C. Kress et al. and a letter of Mar. 20, 2002 to Andrew Wechsler, editor of Journal of Thoracic and Cardiovascular Surgery from David C. Kress, which are incorporated herein by reference.

### EXAMPLE 4

A probe embodying the present invention was fabricated with a tissue contact made by cutting socket boards which are commercially available from Newark Electronics. The particular sockets used are described as low-profile dip sockets and 3-level rap post dip sockets. Both of the aforementioned sockets are illustrated on page 18 of the Newark Electronics catalog, incorporated herein by reference (date unknown). The multi-conductor cable used is also commercially available from Newark Electronics. The particular types of wiring used are shown on page 23 of the Newark Electronics catalog (date unknown).

1. A transmurality evaluation apparatus or probe comprising:
a handheld elongate housing having a first end and a second end and defining an interior passage therebetween;
wiring having electrically coupled first and second ends and positioned within the housing passage and passing therethrough;
at least one tissue contact electrically coupled to the first end of the wiring adjacent the first end of the housing; and
a terminal connector electrically coupled to the second end of the wiring.

2. The transmurality evaluation apparatus as set forth in claim 1, wherein the terminal connector is adapted to electrically couple the wiring to at least one of an implantable cardiac device programmer and a pacemaker programmer.

3. The transmurality evaluation apparatus as set forth in claim 1, wherein the terminal connector is adapted to electrically couple the wiring to a telemetry recording device.

4. The transmurality evaluation apparatus as set forth in claim 1, wherein the terminal connector is adapted to electrically couple the wiring to an electrocardiogram strip recorder.

5. The transmurality evaluation apparatus as set forth in claim 1, wherein the terminal connector is adapted to electrically couple the wiring to a cardiac pacing device.

6. The transmurality evaluation apparatus as set forth in claim 1, wherein the handheld elongate housing is dimensioned to be received within a thoracic surgical port.

7. The transmurality evaluation apparatus as set forth in claim 1, wherein the wiring is electrically insulated.

8. The transmurality evaluation apparatus as set forth in claim 1, wherein the at least one tissue contact is adapted to contact cardiac muscle tissue.

9. The transmurality evaluation apparatus as set forth in claim 1, wherein the at least one tissue contact comprises at least two tissue contacts.

10. The transmurality evaluation apparatus as set forth in claim 1, wherein the at least one tissue contact comprises six or fewer tissue contacts.

11. The transmurality evaluation apparatus as set forth in claim 1, wherein the at least one tissue contact comprises at least two tissue contacts arranged in rows and columns.

12. The transmurality evaluation apparatus as set forth in claim 1, wherein the at least one tissue contact comprises at least two tissue contacts arranged linearly.

13. A method for assessing the transmurality of a lesion comprising:
providing a probe adapted to sense an electrical signal from tissue;
contacting a first region of tissue with the probe;
establishing a first electrogram of the first region of tissue;
ablating a second region of tissue to form an ablated lesion;
contacting the first region of tissue with the probe;
establishing a second electrogram of the first region of tissue; and
comparing the first and second electrograms to assess transmurality of the ablated lesion.

14. The method as set forth in claim 13, wherein contacting the first region of tissue includes epicardially contacting the first region of tissue.

15. The method as set forth in claim 13, wherein ablating a second region of tissue includes ablating a second region of tissue to treat at least one of atrial fibrillation and atrial flutter.

16. The method as set forth in claim 13, further comprising ablating the second region of tissue responsive to comparing the first and second electrograms.

17. The method as set forth in claim 13, wherein the first and second regions of tissue comprises muscle tissue.

18. The method as set forth in claim 13, wherein the first and second regions of tissue comprise cardiac muscle tissue.

19. The method as set forth in claim 13, wherein the first region of tissue comprises at least a portion of a pulmonary vein muscle sleeve, and wherein the second region of tissue comprises at least a portion of the left atrium.

20. The method as set forth in claim 13, wherein the first region of tissue comprises at least one of a pulmonary vein muscle sleeve and at least a portion of the left atrium.

21. The method as set forth in claim 13, wherein the second region of tissue comprises at least one of a pulmonary vein muscle sleeve and at least a portion of the left atrium.

22. A method for assessing the transmurality of a lesion comprising:
providing a probe adapted to sense an electrical signal from tissue, the probe comprising:
a housing having a first end and a second end and defining an interior passage therebetween,
wiring positioned to electrically couple the housing first and second ends and positioned within the housing passage and passing therethrough,
a tissue contact electrically coupled to the first end of the wiring adjacent the first end of the housing, and
a terminal connector electrically coupled to the second end of the wiring;
contacting a first region of tissue with the probe;
establishing a first electrogram of the first region of tissue;
ablating a second region of tissue to form an ablated lesion;
contacting the first region of tissue with the probe;
establishing a second electrogram of the first region of tissue; and
comparing the first and second electrograms to assess transmurality of the ablated lesion.

23. A method for manufacturing a transmurality evaluation apparatus comprising:
providing a handheld elongate housing having a first end and a second end and defining an interior passage therebetween;
positioning wiring having a first end electrically coupled to a second end in the interior passage to electrically couple the housing first end to the housing second end;
electrically coupling a tissue contact to the wiring first end adjacent the housing first end; and
electrically coupling a terminal connector to the wiring second end.

24. The method as set forth in claim 23, wherein the wiring is formed by winding a plurality of wires around each other.

25. The method as set forth in claim 23, wherein electrically coupling a tissue contact includes electrically coupling at least two tissue contacts to the electrical wiring.

26. The method as set forth in claim 23, wherein electrically coupling a tissue contact includes electrically coupling six or fewer tissue contacts to the electrical wiring.

27. The method as set forth in claim 23, wherein electrically coupling a tissue contact includes electrically coupling at least two tissue contacts in an arrangement of rows and columns.

28. The method as set forth in claim 23, wherein electrically coupling a tissue contact includes electrically coupling at least two tissue contacts arranged linearly.

29. The method as set forth in claim 23, further comprising electrically coupling the terminal connector to at least one of an implantable cardiac defibrillator programmer and a pacemaker programmer.

30. The method as set forth in claim 23, further comprising electrically coupling the terminal connector to an electrocardiogram strip recorder.

31. The method as set forth in claim 23, further comprising electrically coupling the terminal connector to a telemetry recording device.

32. The method as set forth in claim 23, further comprising electrically coupling the terminal connector to a cardiac pacing device.
